(19) United States
(12) Patent Application Publication
(10) Pub. No.: US 2024/0390638 A1
(43) Pub. Date: Nov. 28, 2024

(54) VARIABLE STIFFNESS CATHETER LINERS

(71) Applicant: SCIENTIA VASCULAR, INC., West Valley City, UT (US)

(72) Inventors: Nolan LESUEUR, West Valley City, UT (US); William Rigby ECCLES, Centerville, UT (US); Richard F. MAGGIO, Park City, UT (US); Edward J. SNYDER, Park City, UT (US)

(21) Appl. No.: 18/667,495

(22) Filed: May 17, 2024

Related U.S. Application Data
(60) Provisional application No. 63/468,203, filed on May 22, 2023.

(51) Int. Cl. A61M 25/00

(52) U.S. Cl. CPC A61M 25/0045; A61M 25/0052; A61M 25/0053; A61M 2205/0216; A61M 2205/025; A61M 2207/10

(57) ABSTRACT

Disclosed are embodiments for catheters and catheter liners including grooves on the inner surface of the liner. The grooves may improve the flexibility of the catheter while retaining a good stiffness profile. Also disclosed are methods for introducing the grooves on the inner surface of the liner.

### CROSS-REFERENCE TO RELATED APPLICATIONS

This application claims priority to and the benefit of U.S. Provisional Patent Application No. 63/468,203, filed on May 22, 2023 and titled “Techniques for Variable Stiffness Catheter Liners,” which is incorporated herein by reference in its entirety.

### BACKGROUND

Interventional devices such as guidewires and catheters are frequently utilized in the medical field to perform delicate procedures deep within the human body. Typically, a catheter is inserted into a patient's femoral, radial, carotid, or jugular vessel and navigated through the patient's vasculature to the heart, brain, or other targeted anatomy as required. Often, a guidewire is first routed to the targeted anatomy, and one or more catheters are subsequently passed over the guidewire and routed to the targeted anatomy. Once in place, the catheter can be used to deliver drugs, stents, embolic devices, radiopaque dyes, or other devices or substances for treating the patient in a desired manner.

In many applications, such an interventional device must be angled through the tortuous bends and curves of a vasculature passageway to arrive at the targeted anatomy. For example, directing a guidewire and/or catheter to portions of the neurovasculature requires passage through the internal carotid artery and other tortuous paths. Such an interventional device requires sufficient flexibility, particularly near its distal end, to navigate such tortuous pathways. However, other design aspects must also be considered. For example, the interventional device must also be able to provide sufficient torquability (i.e., the ability to transmit torque applied at the proximal end all the way to the distal end), pushability (i.e., the ability to transmit axial push to the distal end rather than bending and binding intermediate portions), and structural integrity for performing intended medical functions.

In addition, conventional catheter devices sometimes utilize different materials to provide a gradient in bending stiffness from proximal end to distal end. However, whenever there is a transition between materials of differing stiffness, the bending, axial, and torsional stiffness profiles of the device includes an abrupt step change. Such abrupt changes in bending stiffness are undesirable because they can concentrate mechanical stresses at particular locations, cause kink points, disrupt the smooth movement and bending of the device, and complicate navigation in tortuous vasculature.

Accordingly, there exist several limitations in the field of catheter devices, and there is an ongoing need, for example, for devices that improve axial and/or torsional response, the distribution of bending forces, and/or are capable of providing a smooth bending stiffness profile.

### SUMMARY

The present disclosure relates to catheters that exhibit effective flexibility and an effective stiffness profile. In one embodiment, the catheter includes a liner, a scaffold disposed about the liner, and an outer member disposed about the scaffold and liner. The inner surface of the liner includes one or more grooves. The grooves can comprise excavations or indentations of the liner wall. The flexibility of the liner is thereby increased, relative to a liner of otherwise similar construction, as less material is present in portions of the liner and thus the liner wall is less resistant to bending motions of the liner. The improved flexibility may be achieved while still retaining good pushability of the liner.

The grooves may form a groove pattern, such as helical grooves, that extend over at least a portion of the liner. Other groove patterns include groove rings and short disconnected grooves. The groove pattern may also be altered over the length of the liner to provide a variable, customized, and/or more desirable stiffness profile.

Also disclosed are methods for introducing grooves on the inner surface of the liner. In one embodiment, the liner material is extruded over a mandrel, the mandrel comprising ridges with a complementary shape to the desired groove pattern. Subsequent removal of the mandrel reveals a groove pattern on the inner surface of the liner. In another embodiment, the grooves are formed by cutting the inner surface of the liner after the liner has been formed on the mandrel.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

### BRIEF DESCRIPTION OF THE DRAWINGS

Various objects, features, characteristics, and advantages of the invention will become apparent and more readily appreciated from the following description of the embodiments, taken in conjunction with the accompanying drawings and the appended claims, all of which form a part of this specification. In the Drawings, like reference numerals may be utilized to designate corresponding or similar parts in the various Figures, and the various elements depicted are not necessarily drawn to scale, wherein:

FIG. 1 illustrates an overview of an example catheter system, including a catheter and hub;

FIG. 2 illustrates a detailed view showing various sections of the catheter of the catheter system;

FIG. 3 illustrates a proximal-distal cross section of a portion of the catheter liner, including grooves on the inner surface of the liner;

FIGS. 4A through 4C illustrate cross sections of the catheter liner, the cross sections transverse to a proximal-distal axis of the liner, showing various groove widths;

FIG. 5 illustrates a proximal-distal cross section of a portion of the catheter liner, including intersecting helical grooves on the inner surface of the liner;

FIG. 6 illustrates a proximal-distal cross section of a portion of the catheter liner, including groove rings distributed about the proximal-distal axis of the liner;

FIG. 7 illustrates a proximal-distal cross section of a portion of the catheter liner, including a groove pattern of short discrete grooves on the inner surface of the liner;

FIG. 8 illustrates a proximal-distal cross section of a proximal portion and a distal portion of the catheter liner, wherein the proximal portion of the liner includes a helical groove having a greater pitch than the pitch of a helical groove at the distal portion of the liner;

FIG. 9 illustrates a proximal-distal cross section of a proximal portion and a distal portion of the catheter liner, wherein the proximal portion of the liner includes a helical groove having a greater width than the width of a helical groove at the distal portion of the liner;

FIG. 10 illustrates a mandrel including a helical ridge;

FIG. 11 illustrates a proximal-distal cross section of a portion of the catheter liner disposed about a mandrel including a helical ridge;

FIG. 12 illustrates a first end of a mandrel including an excision tool.

### DETAILED DESCRIPTION

### Example Catheter Overview

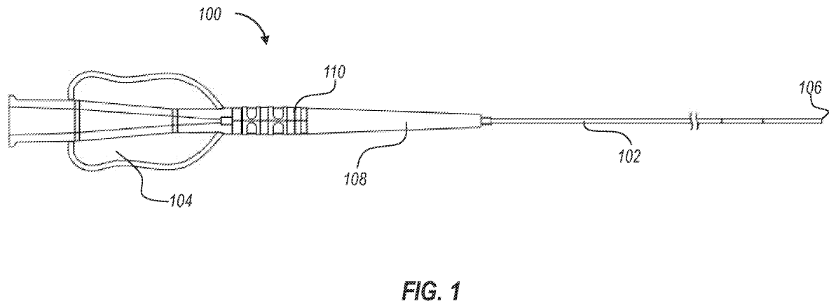

FIG. 1 is an overview of an example catheter device 100 that includes features, described in more detail below, that provide one or more of improved axial responsiveness, improved distribution of bending forces, and/or a smooth device bending stiffness profile.

The catheter device 100 includes a catheter 102 connected to a hub 104 at a proximal end and extending therefrom to a distal end 106. The catheter 102 may be coupled to the hub 104 using adhesive, a friction fit, through insertion molding, and/or other appropriate attachment means. A strain-relief member 108 may also be disposed over the proximal section of the catheter 102 near the hub 104. The strain-relief member 108 has an outer diameter that substantially matches the adjacent section of the hub 104. The strain-relief member 108 may extend for a distance from the hub 104 with a substantially constant outer diameter before tapering distally to the end where the catheter 102 emerges and extends farther distally. The strain-relief member 108 may include a strain-relief indentation pattern 110, disposed at the section of substantially constant outer diameter, that functions to provide additional flexibility to the strain-relief member 108 and/or to provide surface features for enhancing user grip and tactile engagement.

The working length of the catheter 102 (i.e., the distance between the distal end of the strain-relief member 108 and the distal end 106 of the catheter 102 may vary according to particular application needs. As an example, the catheter 102 may have a working length of about 50 cm to about 200 cm, though shorter or longer lengths may be utilized where appropriate. The catheter size (typically referring to the inner diameter/lumen size) may also vary according to particular application needs. Examples include 0.010 inches, 0.013 inches, 0.017 inches, 0.021 inches, 0.027 inches, 0.030 inches, 0.035 inches, 0.038 inches, 0.045 inches, 0.065 inches, 0.085 inches, 0.100 inches, or a range including any two of the foregoing values as endpoints. The inside diameter of the catheter 102 can taper from a smaller distal portion to a larger proximal portion. Smaller or larger sizes may be utilized in some applications as appropriate.

Although the distal section of the catheter 102 is shown in this example as having a straight shape, other embodiments may include a shaped distal tip. For example, the distal section of the catheter 102 may have an angled shape, a curved shape (e.g., 45 degree angle, 90 degree angle, J shape, etc.), a compound curved shape, or other appropriate angled or bent shape as known in the art.

The catheter device 100 described herein may be utilized for a variety of interventional applications, most commonly in cardiovascular, peripheral vascular, and neurovascular interventional procedures. Examples include accessing distal anatomy, crossing vessel lesions or blood clots, ischemic treatments, delivering therapeutic agents (e.g., embolic coils or other embolic agents), injecting diagnostic agents (e.g., contrast media or saline), retrieval applications, aspiration applications, or other applications where microcatheter use is beneficial.

The outer surface of the catheter 102 may be coated with an appropriate coating material, such as a hydrophilic coating, to make the surface more lubricious. Examples of such materials include polyether block amide (PEBA), polyether ether ketone (PEEK), or other polyaryl ether ketone (PAEK). The coating material may cover substantially all of the working length of the catheter 102, or a portion thereof. For example, the coating material may be applied to the distalmost 30% to 80% of the working length of the catheter 102.

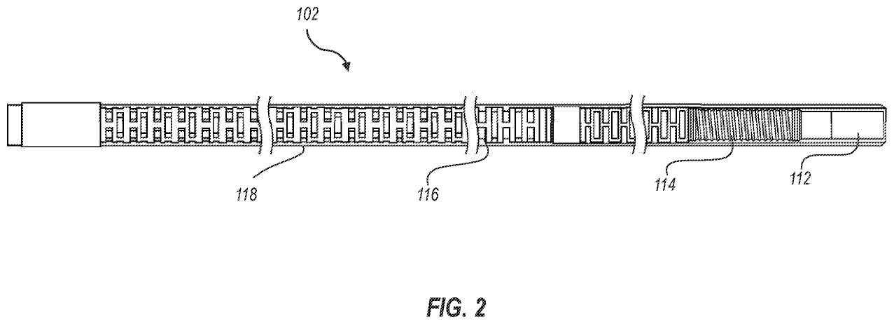

FIG. 2 illustrates a detailed view of an exemplary catheter 102, better showing some of the internal components and different longitudinal sections that may comprise the catheter 102. As shown, the catheter 102 includes an inner liner 112 comprising a tube and that defines the inner lumen of the device. The liner 112 may be formed from polytetrafluoroethylene (PTFE) and/or other appropriate polymer, plastic, or metal. The liner 112 may be formed by extruding an appropriate polymer over a mandrel, copper wire, or other substrate.

A scaffold, comprising a coil, braid, or shaft, or a combination of the foregoing, is typically positioned over the liner 112. For example, a coil 114 may be positioned on the liner 112 near the distal end 106 of the device. A microfabricated shaft 116 (also referred to herein as “inner shaft”) may also be positioned on the liner 112. The coil 114 may be attached to or positioned next to the inner shaft 116, the inner shaft 116 extending proximally therefrom. An outer layer 118, formed from one or more polymer materials, is typically heat shrink laminated over and through the scaffold (i.e., the coil 114 and inner shaft 116), encasing the scaffold while also attaching to the liner 112.

In one embodiment, the coil 114 is formed from stainless steel and the inner shaft 116 is formed from nitinol. These materials, when used in combination with other features described herein, have been found to provide effective axial response, effective distribution of bending forces, and a smooth bending stiffness profile. Other embodiments may utilize one or more different materials for the coil 114, the inner shaft 116, or both. In some embodiments, for example, the inner shaft 116 may include other superelastic alloys and/or one or more polymers such as a polyether block amide (PEBA), polyether ether ketone (PEEK), or other polyaryl ether ketone (PAEK). In some embodiments, the coil 114 may include a superelastic alloy such as nitinol, a radiopaque material, one or more other metals, alloys, or polymers.

The catheter 102 may also include one or more radiopaque marker bands or coils. The radiopaque marker bands or coils are formed from a material more radiopaque than stainless steel. Examples include platinum, iridium, tungsten, other highly radiopaque metals, and alloys thereof. The radiopaque marker bands or coils may provide an indication of the location of the distal end 106 or other portions of the catheter 102 and may assist in the proper positioning of detachable embolic coils or other components deployed through the catheter 102.

The catheter 102 may be configured so that the overall bending stiffness profile transitions from higher stiffness (and less bending flexibility) at the proximal sections to lower stiffness (and greater bending flexibility) at the distal sections. In most applications, it is desirable to give the proximal sections of the device relatively high axial, torsional, and bending stiffness so they can provide good combination of flexibility, pushability, and torquability. Distal sections, however, are often navigated through tortuous vasculature and are thus preferably relatively more flexible in bending. As explained in more detail below, the liner 112 may include a structured inner surface for configuring the bending stiffness of the device 102.

### Flexible Liners

The following description is directed to the structured inner surface of the liner 112. The liner 112 provides the point of contact between a guidewire and the catheter 102 as the catheter 102 is navigated to the targeted anatomical location. The liner 112 may also serve to deliver treatment fluids to the target site. Thus, important characteristics of the liner 112 include good lubricity (for good trackability over the guidewire) and fluid integrity.

Catheter liners 112 have been a source of undesirable stiffness in the catheter 102. This is particularly problematic at the distal end 106 of the catheter 102 where the catheter 102 may need to navigate tight turns within the patient's vasculature. In some examples, catheter manufacturers have eliminated the liner 112 from the distal section of the catheter 102 to improve catheter 102 flexibility at the distal end 106. However, such catheters often encounter kinking issues at the distal portion of the device, hampering efficient navigation of the catheter and increasing operation complexity and risk. Removal of the liner 112 from distal portions also removes the ability to effectively transmit fluids to the distal end of the catheter.

Other catheters may include a liner 112 of a stiffer material at a proximal portion of the catheter 102 that transition to a liner 112 of a different, more flexible material at a more distal portion of the catheter 102. But these catheters 102 also encounter kinking issues and are more costly to manufacture. Moreover, the transition point is subject to increased risk of stress buildup, breakage, and/or disruptions in liner integrity.

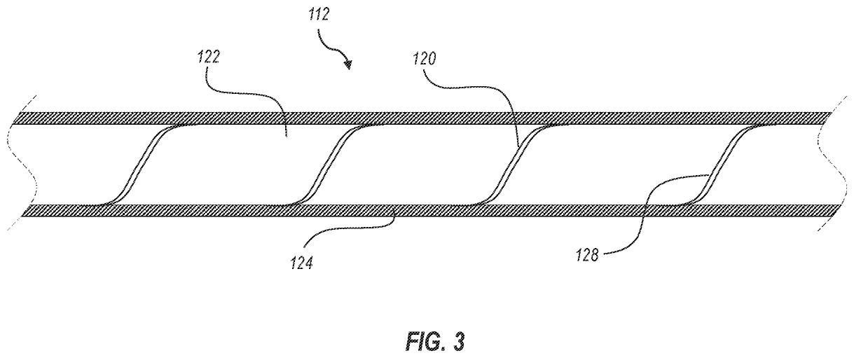

The catheter liners 112 disclosed herein can be configured to exhibit improved flexibility while retaining sufficient kink resistance and tensile strength. This can be accomplished through the inclusion of one or more grooves 120. FIG. 3 illustrates a cross sectional view of a portion of an exemplary liner 112 including grooves 120 on the inner surface 122 of the liner 112. The grooves 120 may comprise cuts or indentations made in the inner surface 122 of the liner 112, resulting in relatively less material in the wall 124 of the liner 112. The exclusion of material at particular locations along the liner 112 enables the liner 112 to bend to a greater degree, increasing the flexibility of the device 100.

Preferably, the grooves 120 extend from the inner surface 122 of the liner 112 to a depth of only a portion of the total wall thickness of the liner 112, such that the fluid integrity of the liner 112 is maintained. For example, the depth of the grooves 120 may be as great as 10%, 20%, 30%, 40%, 50%, 60%, 70%, 80%, or 90% of the thickness of the liner wall 124. The width of the grooves 120 may extend within a range of about 5°, about 25°, about 45°, about 65°, about 85°, about 105°, or about 120° of the circumference of the inner surface of the liner 112. Groove depths and widths within the foregoing can beneficially provide the increased flexibility effects while maintaining sufficient liner integrity.

It will be appreciated that the greater the depth or width of the groove 120, the greater the flexibility that is imparted to the liner 112. As the size of the groove 120 increases there is less material to resist bending motions of the liner 112. It will also be appreciated that relatively larger sizes of grooves 120 are associated with reduced stiffness and pushability of the liner 112.

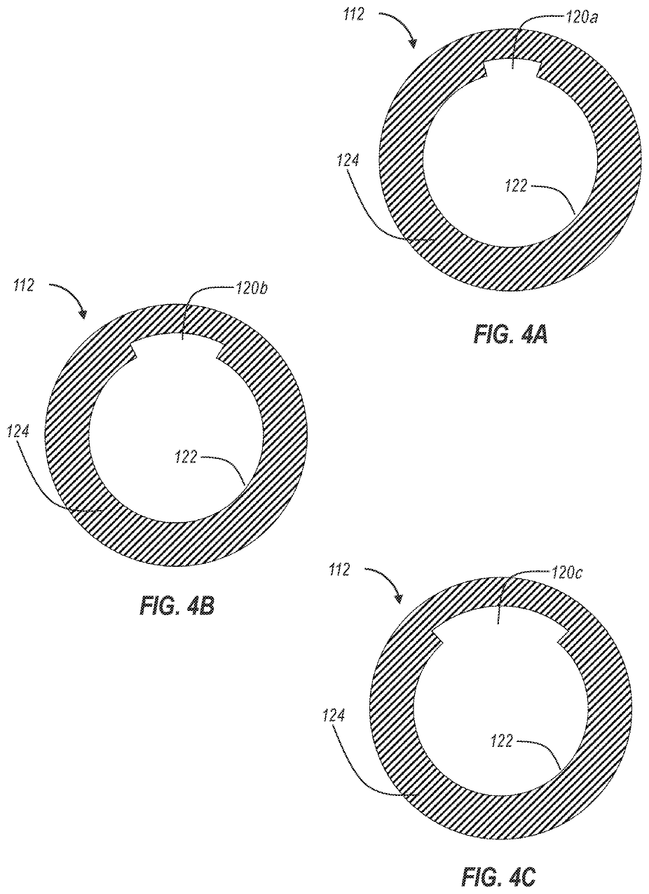

FIGS. 4A-4C illustrate a set of liner cross sections, the plane of the cross sections being oriented perpendicular to a proximal-distal axis of the catheter 102. The liner cross sections of FIGS. 4A-4C depict grooves 120 of various widths, including a relatively small-width groove 120a (FIG. 4A), a relatively medium-width groove 120b (FIG. 4B), and a relatively large-width groove 120c (FIG. 4C). Thus, it would be expected that the groove 120b illustrated in FIG. 4B would impart greater flexibility to the liner 112 compared to the groove 120a of FIG. 4A, with the groove 120c of FIG. 4C imparting greater flexibility to the liner 112 than either that of FIG. 4B or FIG. 4A.

The one or more grooves 120 may form a groove pattern that is repeated over a portion of the liner 112. In one embodiment, the groove pattern comprises a helical groove 128, as shown in FIG. 3. The helical groove 128 is advantageous in that the liner 112 remains equally bendable in all directions (i.e., preferred bending directions are minimized) while maintaining adequate tensile strength for good pushability.

The tensile strength and stiffness of the liner 112 may be altered by adjusting the pitch 130 (see FIG. 8) of the helical groove 128 (in addition to adjusting the width and/or depth of the grooves). Thus, as the pitch 130 of the helical groove 128 is decreased, the stiffness of the liner 112 also decreases while also decreasing the tensile strength. Conversely, as the pitch 130 of the helical groove 128 is increased, so too is the stiffness and tensile strength of the 112.

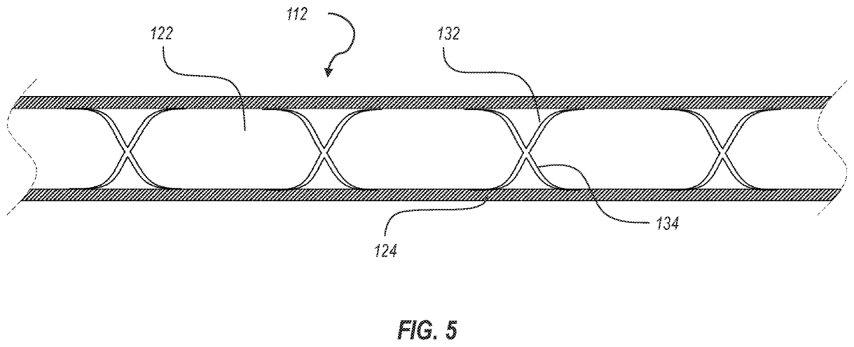

As shown in FIG. 5, the groove pattern may comprise two helical grooves 128, including first 132 and second 134 helical grooves. The first 132 and second 134 helical grooves may be substantially parallel along the inner surface of the liner 112, such that the pitch 130 of the first helical groove 132 is substantially similar to the pitch 130 of the second helical groove 134. Alternatively, the first 132 and second 134 helical grooves may intersect. The rotation direction of the first helical groove 132 may be oriented opposite that of the second helical groove 134, the groove pattern resembling a braid, such as that shown in FIG. 5. In another embodiment, the pitches of the first 132 and second 134 helical grooves may be oriented in the same direction but have different magnitudes. In still other embodiments, the groove pattern may comprise three, four, five, or more helical grooves 128.

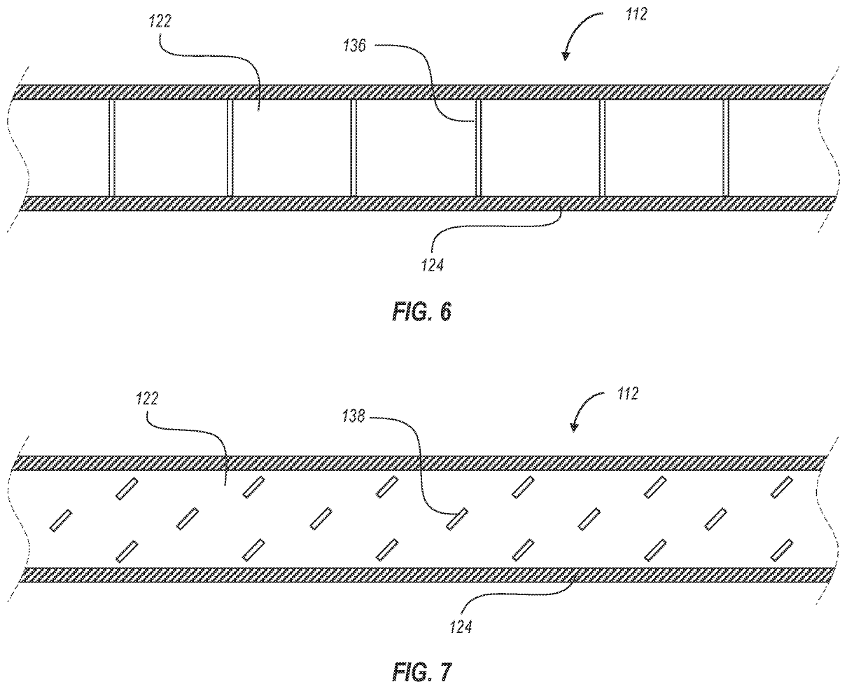

The groove(s) 120 may extend along a substantial portion of the liner 112. For example, a helical groove 128 may extend from a proximal end to a distal end of the liner 112. The groove pattern may also take other configurations wherein the groove 112 extends along only a small length of the liner 112. For example, the groove pattern may comprise a series of groove rings 136 disposed along a proximal-distal axis of the liner 112, as shown in FIG. 6. The groove pattern may also comprise shorter disconnected grooves 138, such as those shown in FIG. 7.

The flexibility along the length of the catheter 102 may be configured by adjusting aspects of the grooves 120 along the liner 112. It will also be appreciated that there are many adjustments to the groove pattern that may increase the flexibility of the liner 112, of which only a few are described below.

Generally, the flexibility of the distal portion 140 of the liner 112 may be increased where grooves 120 cover the inner surface of the distal portion 140 to a greater extent than the inner surface of other portions of the liner 112. For example, FIG. 8 illustrates an embodiment of a proximal portion 142 and a distal portion 140 of a liner 112. The liner 112 includes a helical groove 128, wherein the pitch 130 of the helical groove 128 is smaller at the distal portion 140 of the liner 112 than at the proximal portion 142. Thus, the distal portion 140 of the liner 112 will exhibit greater flexibility compared to the proximal portion 142 of the liner 112, as the distal portion 140 includes less material in the wall 124 of the liner 112 to resist bending motions of the liner 112. The pitch 130 of the helical groove 128 may be adjusted in discrete sections over the length of the liner 112 or may be adjusted continuously along the liner 112.

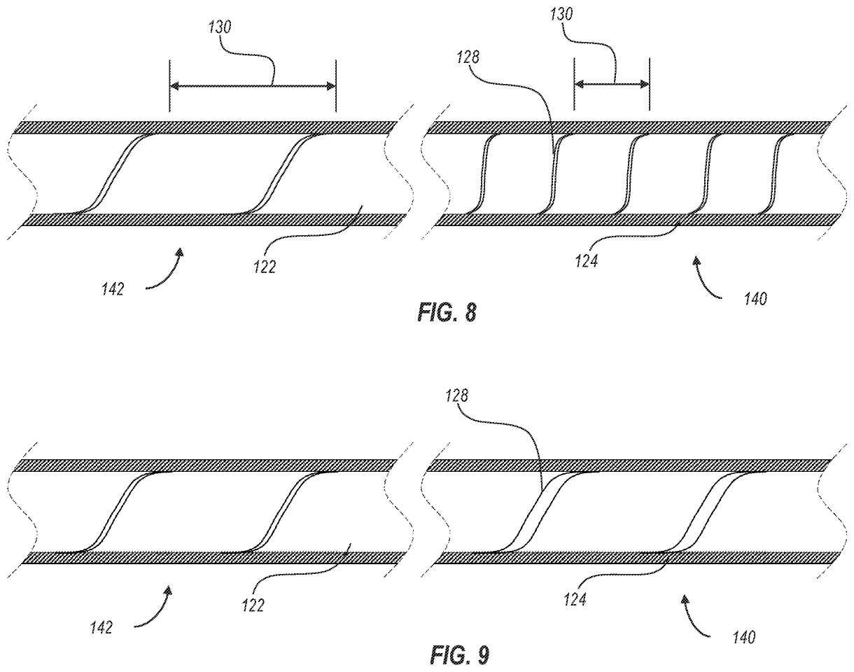

The flexibility of the liner 112 may additionally or alternatively be adjusted by varying the width and/or depth of the grooves 120. For example, the grooves 120 may also be wider and/or deeper at a distal portion 140 compared to a proximal portion 142 of the liner 112. FIG. 9 illustrates an embodiment comprising a helical groove 128 wherein the groove 120 is wider at the distal portion 140 compared to the groove 120 at the proximal portion 142. In other embodiments the depth of the grooves 120 may be greater at the distal portion 140 compared to the proximal portion 142 of the liner 112. Similar to the embodiments described above, the width and depth of the grooves 120 may also be adjusted in discrete sections or continuously along the length of the liner 112.

As illustrated herein, while the inner surface 122 of the liner 112 may be formed with grooves 120, the outer surface of the liner 112 is preferably substantially continuous (i.e., is substantially smooth and does not include grooves or other discontinuities). This allows for good engagement between the liner 112 and the overlying structures of the catheter 102, such as with the shaft 116.

### Methods of Forming Liner Grooves

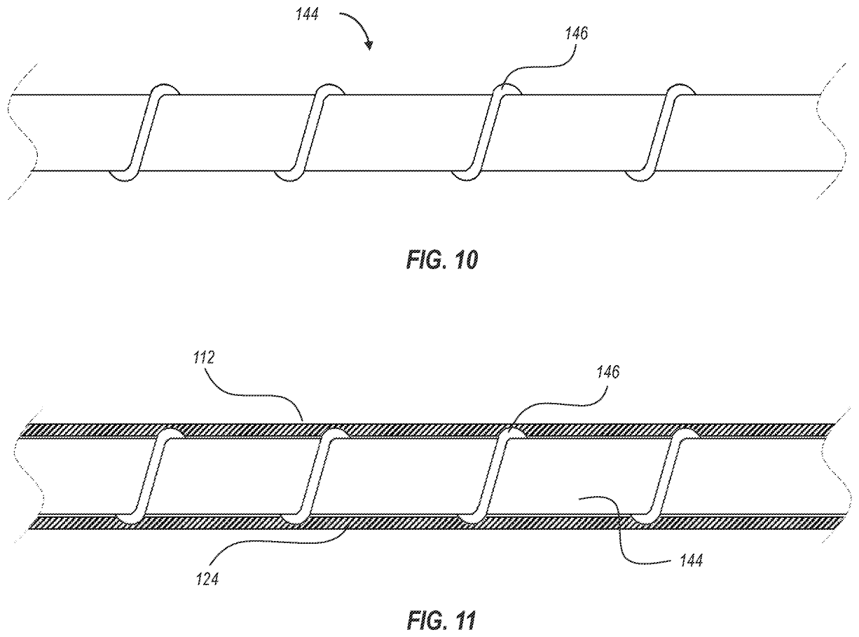

The grooves 120 of the inner surface 122 of the liner 112 may be introduced through multiple methods. In one embodiment, liner material is extruded or otherwise placed over a mandrel 144. FIG. 10 illustrates an exemplary mandrel 144 over which liner material may be extruded. The mandrel 144 may include one or more ridges that correspond to the groove pattern desired on the inner surface 122 of the liner 112. The one or more ridges may protrude from the mandrel or may comprise a separate structure that is placed about (and optionally affixed to) the mandrel 144 before the liner material is extruded over the mandrel 144.

The liner material may be shaped to conform to the shape of the mandrel 144, such that the surface of the mandrel 144 and ridges and the inner surface 122 of the liner 112 have a complementary shape. This may be accomplished by heating the liner material after the liner material has been extruded over the mandrel 144. Particularly, the liner material may be heated when the outer member 118 is heat shrink laminated over a coil 114, shaft 116, and/or the liner 112. Once the liner 112 has conformed to the shape of the mandrel 144 and ridges, it is removed from the mandrel 144 resulting in a liner 112 including grooves 120 that complement the mandrel ridges.

For example, the mandrel 144 may include a helical ridge 146, such as that shown in FIG. 10, for producing a helical groove 128 on the inner surface 122 of the liner 112. FIG. 11 illustrates a liner 112 extruded over the mandrel 144 of FIG. 10, the liner 112 being formed around the mandrel 144 and its helical ridge 146 to produce a helical groove 128 on the inner surface 122 of the liner 112.

In embodiments wherein the mandrel 144 includes one or more helical ridges 146 with a uniform pitch, the mandrel 144 may be removed simply by pulling the mandrel 144 from the liner 112 and twisting the mandrel 144 as though disengaging a threaded attachment. Removing the mandrel 144 in this manner ensures that the helical ridges 146 will not distort the groove pattern or otherwise compromise the integrity of the liner 112. This manner of removal may also suffice in cases where the width of the helical ridge 146 increases unidirectionally along the mandrel 144.

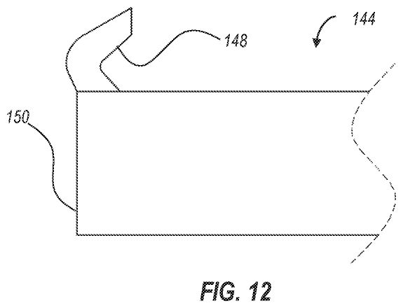

In other embodiments, the inner surface 122 of the liner 112 may be cut after the liner 112 is formed. For example, in cases where the liner 112 is formed over a mandrel 144 the mandrel 144 may include an excision tool 148, such as a small blade. FIG. 12 illustrates a mandrel 144 including an exemplary excision tool 148. The excision tool 148 may be located at a first end 150 of the mandrel 144. After the liner 112 has been formed over the mandrel 144 the mandrel 144 is then removed in a direction from the first end 150 of the mandrel 144 toward the second end, such that the excision tool 148 of the mandrel 144 is pulled along the length of the liner 112.

The excision tool 148 is oriented on the mandrel 144 such that as the mandrel 144 is pulled through the liner 112 the excision tool 148 comes into contact with at least a portion of the inner surface 122 of the liner 112. As the excision tool 148 passes through the liner 112 it removes material from the inner surface 122 of the liner 112. The mandrel 144 may be rotated as it is removed from the liner 112 so as to form a helical groove or other groove pattern based on the cut.

After the mandrel 144 has been removed from the liner 112, the mandrel 144 may be reinserted one or more times into the liner 112 so as to form additional groove patterns on the inner surface 122 of the liner 112.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

1. An intravascular device, comprising:
a scaffold;
a liner extending through the scaffold such that an outer surface of the liner engages with an inner surface of the scaffold; and
an outer polymer layer disposed over the scaffold and the liner,
wherein an inner surface of the liner comprises one or more grooves.

2. The device of claim 1, wherein a stiffness of the liner varies along a proximal-distal length of the liner.

3. The device of claim 1, wherein a wall thickness of the liner varies along a proximal-distal length of the liner.

4. The device of claim 1, wherein the inner surface of a distal portion of the liner comprises grooves in a greater proportion than the inner surface of a proximal portion of the liner.

5. The device of claim 1, wherein a depth of the one or more grooves is greater at a distal portion of the liner than at a proximal portion of the liner.

6. The device of claim 1, wherein a width of the one or more grooves is greater at a distal portion of the liner than at a proximal portion of the liner.

7. The device of claim 1, wherein the liner comprises polytetrafluoroethylene (PTFE).

8. The device of claim 1, wherein the one or more grooves form a groove pattern on the inner surface of the tube.

9. The device of claim 8, wherein the groove pattern comprises one or more groove rings disposed about a proximal-distal axis of the tube.

10. The device of claim 8, wherein the groove pattern comprises a helical groove.

11. The device of claim 10, wherein a pitch of the helical groove varies distally along the liner.

12. The device of claim 11, wherein the pitch of the helical groove decreases distally along the liner.

13. The device of claim 8, wherein the groove pattern comprises a first helical groove and a second helical groove.

14. The device of claim 13, wherein the first helical groove has a pitch oriented opposite that of the second helical groove.

15. The device of claim 1, wherein the scaffold comprises a shaft, braid, and/or coil.

16. The device of claim 1, wherein the outer polymer layer comprises polyether block amide (PEBA), polyether ketone (PEEK), and/or other polyaryl ether ketone (PAEK).

17. An intravascular device, comprising:
a scaffold comprising a shaft, and a coil, wherein the shaft includes a plurality of fenestrations and wherein the coil is disposed distal of the shaft;
a liner extending through the scaffold such that an outer surface of the liner engages with an inner surface of the scaffold; and
an outer polymer layer disposed over the scaffold and the liner,
wherein an inner surface of the liner comprises one or more grooves,
wherein the one or more grooves form a helical groove pattern in the inner surface of the liner.

18. A method of making an intravascular device, the method comprising:
a) disposing a liner over a mandrel, the mandrel comprising one or more ridges that complement a pattern of grooves to be formed on an inner surface of the liner;
b) disposing a scaffold over the liner and the mandrel;
c) disposing a polymer layer over the scaffold, the liner, and the mandrel to form a catheter assembly, and
d) heating the catheter assembly along a length of the polymer layer.

19. The method of claim 18, wherein the scaffold includes fenestrations or gaps and wherein the polymer layer and the liner material join during the heating step through the fenestrations or gaps to encapsulate the scaffold.

20. The method of claim 18, wherein the ridges of the mandrel form a helical pattern of grooves in the liner, and wherein the mandrel is removed by twisting relative to the liner.
